(19) United States
(12) Patent Application Publication
(10) Pub. No.: US 2025/0006359 A1
(43) Pub. Date: Jan. 2, 2025

(54) METHODS FOR CONFIGURING IMAGING PARAMETERS

(71) Applicant: WUHAN UNITED IMAGING HEALTHCARE CO., LTD., Wuhan (CN)

(72) Inventor: Jiao BA, Wuhan (CN)

(73) Assignee: WUHAN UNITED IMAGING HEALTHCARE CO., LTD., Wuhan (CN)

(21) Appl. No.: 18/884,461

(22) Filed: Sep. 13, 2024

Related U.S. Application Data
(63) Continuation of application No. PCT/CN2022/134351, filed on Nov. 25, 2022.

(30) Foreign Application Priority Data
Apr. 2, 2022 (CN) 202210341722.8

(51) Int. Cl. G16H 40/63; A61B 6/00; A61B 90/96; G16H 10/60

(52) U.S. Cl. CPC G16H 40/63; A61B 90/96; G16H 10/60; A61B 6/545

(57) ABSTRACT

Embodiments of the present disclosure provides a method for configuring imaging parameters. The method includes obtaining a user identifier of a user to be examined and one or more device parameters to be set of an imaging device; determining target values of the one or more device parameters based on the user identifier; and performing parameter configuration on the imaging device based on the target values of the one or more device parameters. According to the present disclosure, the efficiency of examining a patient using a medical imaging device may be improved by determining recommended parameters of the imaging device.

### CROSS-REFERENCE TO RELATED APPLICATIONS

The present disclosure is a Continuation of International Patent Application No. PCT/CN2022/134351, filed on Nov. 25, 2022, which claims priority to Chinese Patent Application No. 202210341722.8, filed on Apr. 2, 2022, the entire contents of which are hereby incorporated by reference.

### TECHNICAL FIELD

The present disclosure relates to the field of medical imaging, and in particular, to methods for configuring imaging parameters.

### BACKGROUND

With the development of medical imaging technology, the medical imaging device is playing an increasingly important role in the auxiliary diagnosis of diseases. By adjusting parameters of the medical imaging device, lesion regions of different patients may be scanned and imaged, thereby accurately obtaining lesion information for different patients and assisting doctors in diagnosing diseases.

When using a medical imaging device to re-examine a patient, the doctor often needs to spend time re-inputting the personal information of the patient and imaging parameters of the medical imaging device, which is time-consuming for both doctor and patient. Therefore, it is desirable to provide a method for configuring imaging parameters to quickly determine the imaging parameter of the medical imaging device.

### SUMMARY

One or more embodiments of the present disclosure provide a method for configuring imaging parameters. The method may include obtaining a user identifier of a user to be examined and one or more device parameters to be set of an imaging device; determining target values of the one or more device parameters based on the user identifier; and performing parameter configuration on the imaging device based on the target values of the one or more device parameters.

One or more embodiments of the present disclosure provide a system for configuring imaging parameters. The system may include an acquisition module, a determination module, and a parameter configuration module. The acquisition module may be configured to obtain a user identifier of a user to be examined and one or more device parameters to be set of an imaging device. The determination module may be configured to determine target values of the one or more device parameters based on the user identifier. The parameter configuration module may be configured to perform parameter configuration on the imaging device based on the target values of the one or more device parameters.

One or more embodiments of the present disclosure provide a device for configuring imaging parameters. The device may include at least one processor and at least one storage. The at least one storage may be configured to store computer instructions. The at least one processor may be configured to execute at least a portion of the computer instructions to implement the method for configuring imaging parameters hereinabove.

One or more embodiments of the present disclosure provide a non-transitory computer-readable storage medium. The computer instructions are executed by a processor to implement the method for configuring imaging parameters hereinabove.

### BRIEF DESCRIPTION OF THE DRAWINGS

The present disclosure is further illustrated in terms of exemplary embodiments. These exemplary embodiments are described in detail with reference to according to the drawings. These embodiments are non-limiting exemplary embodiments, in which like reference numerals represent similar structures, and wherein:

FIG. 1 is a schematic diagram illustrating an application scenario of a system for configuring imaging parameters according to some embodiments of the present disclosure;

FIG. 2 is a block diagram illustrating an exemplary system for configuring imaging parameters according to some embodiments of the present disclosure;

FIG. 3 is a flowchart illustrating an exemplary process for configuring imaging parameters according to some embodiments of the present disclosure;

FIG. 4 is a flowchart illustrating an exemplary process for determining target values of device parameters according to some embodiments of the present disclosure;

FIG. 5 is a flowchart illustrating an exemplary process for determining reference values of device parameters according to some embodiments of the present disclosure;

FIG. 6 is a schematic diagram illustrating an association model according to some embodiments of the present disclosure;

FIG. 7 is a flowchart illustrating an exemplary process for determining a target reference user according to some embodiments of the present disclosure; and

FIG. 8 is a schematic diagram illustrating a parameter prediction model according to some embodiments of the present disclosure.

### DETAILED DESCRIPTION

To more clearly illustrate the technical solutions related to the embodiments of the present disclosure, a brief introduction of the drawings referred to the description of the embodiments is provided below. Obviously, the drawings described below are only some examples or embodiments of the present disclosure. Those having ordinary skills in the art, without further creative efforts, may apply the present disclosure to other similar scenarios according to these drawings. Unless obviously obtained from the context or the context illustrates otherwise, the same numeral in the drawings refers to the same structure or operation.

It should be understood that “system,” “device,” “unit” and/or “module” as used herein is a manner used to distinguish different components, elements, parts, sections, or assemblies at different levels. However, if other words serve the same purpose, the words may be replaced by other expressions.

As shown in the present disclosure and claims, the words “one,” “a,” “a kind” and/or “the” are not especially singular but may include the plural unless the context expressly suggests otherwise. In general, the terms “comprise,” “comprises,” “comprising,” “include,” “includes,” and/or “including,” merely prompt to include operations and elements that have been clearly identified, and these operations and elements do not constitute an exclusive listing. The methods or devices may also include other operations or elements.

The flowcharts used in the present disclosure illustrate operations that systems implement according to some embodiments of the present disclosure. It should be understood that the previous or subsequent operations may not be accurately implemented in order. Instead, each step may be processed in reverse order or simultaneously. Meanwhile, other operations may also be added to these processes, or a certain step or several steps may be removed from these processes.

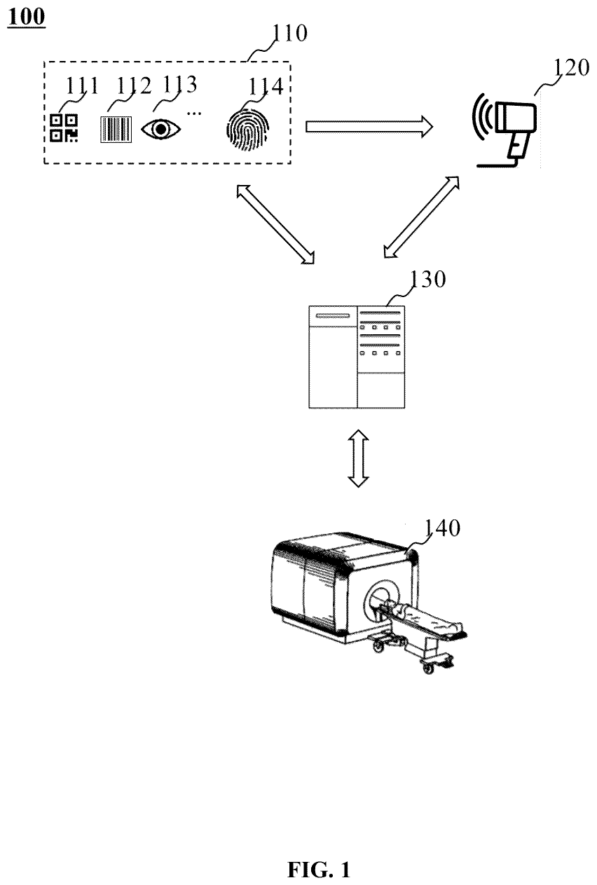

FIG. 1 is a schematic diagram illustrating an application scenario of a system for configuring imaging parameters according to some embodiments of the present disclosure. As shown in FIG. 1, an application scenario 100 may include a detection device 120, a processing device 130, and a medical imaging device 140. In some embodiments, the system for configuring the imaging parameters (referred to as a parameter configuration system) may serve as a part of the processing device 130 used in a service platform for recommending medical imaging device parameters. For example, the system may perform parameter recommendations for the medical imaging device by implementing the methods and/or processes disclosed in the present disclosure. In some embodiments, the system may serve as a part of the medical imaging device 140 for determining the imaging parameters of the medical imaging device 140.

The processing device 130 may communicate with the detection device 120 and the medical imaging device 140 via a network to provide various functions of the online service. In some embodiments, the detection device 120 may scan an identifier 110 to obtain user information corresponding to the identifier 110 and transmit the user information to the processing device 130. In some embodiments, the processing device 130 may obtain information relating to the medical imaging device 140 and process the information relating to the medical imaging device 140 and the user information to determine imaging parameters of the medical imaging device 140 and send the imaging parameters of the medical imaging device 140 to the medical imaging device 140.

The identifier 110 refers to an information carrier configured to record information relating to a user, which may be used as a unique identifier to identify the user. In some embodiments, one user may correspond to at least one identifier 110. In some embodiments, the identifier 110 may be a numeric identifier, for example, the identifier 110 may include a quick response (QR) code 111, a bar code 112, or the like. In some embodiments, the identifier 110 may be a biometric identifier, e.g., an iris 113, a fingerprint 114, a voiceprint, etc., of the user.

The detection device 120 refers to a device configured to perform a detection (e.g., a scan) on the identifier 110. Data and/or information corresponding to the user may be obtained based on the detection of the identifier 110 by the detection device 120. In some embodiments, the detection device 120 may be different devices based on different identifiers 110. For example, when the identifier 110 is a numeric identifier such as the QR code, the bar code, or the like, the detection device 120 may be a scanner gun, a QR code recognizer, a camera, or the like, respectively. For example, when the identifier 110 is a biometric identifier, the detection device 120 may be an iris scanner, a fingerprint scanner, a voice capture device, or the like.

In some embodiments, the processing device 130 may be communicatively connected with the medical imaging device 140 and recommend parameter values of device parameters to the medical imaging device 140. In some embodiments, when the user is a follow-up user, based on the scan on the identifier 110 by the detecting device 120, identity information of the use may be quickly determined, and based on the identity information of the user, historical scan information of the user may be obtained from a database. For example, the user information such as basic information of the user and parameter information of the imaging device at each scan may be stored, and a storage address may be generated as a QR code by QR code encoding software, and the QR code may be added to a diagnostic report of the user. When undergoing a follow-up examination, the user typically brings his or her historical diagnostic report. Doctors may use the detection device 120 to scan the QR code, thereby obtaining the corresponding user information. In some embodiments, the user information includes the historical scan information, and the historical scan information may include historical parameter values of a historical scan. The parameter values of the device parameters of the current scan may be quickly determined based on the historical parameter values. For example, historical parameter values from a historical scan of the same type may be used as the parameter values of the device parameters of the current scan.

The processing device 130 may be configured to process data and/or information of at least one component of the application scenario 100 or an external data source (e.g., a cloud data center). In some embodiments, the processing device 130 may be a single server or group of servers. The group of servers may be centralized or distributed (e.g., the processing device 130 may be a distributed system) and may be dedicated or served simultaneously by other devices or systems. In some embodiments, the processing device 130 may be regional or remote. In some embodiments, the processing device 130 may be implemented on a cloud platform or provided virtually. By way of example only, the cloud platform may include a private cloud, a public cloud, a hybrid cloud, a community cloud, a distributed cloud, an on-premises cloud, a multi-tiered cloud, etc., or any combination thereof.

The medical imaging device 140 may be configured to obtain image data of a scanned object. The scanned object may be a part of an organ or a tissue of the human body, such as the head, the chest, limbs, or the like. The scanned object may also be other living things (e.g., animals) or non-living things (e.g., phantoms). In some embodiments, the medical imaging device 14 may include a unimodal medical imaging device and/or a multimodal medical imaging device. The unimodal medical imaging device may include an ultrasound (US) scanner, an X-ray scanner, a computed tomography (CT) scanner, a magnetic resonance imaging (MRI) scanner, a positron emission computed tomography (PET) scanner), an optical coherence tomography (OCT) scanner, an intravenous ultrasound (IVUS) scanner, a near infrared spectroscopy (NIS) scanner, a far infrared (FIR) scanner, etc., or any combination thereof. The multimodal medical imaging device may include an X-ray-magnetic resonance imaging (X-ray-MRI) scanner, a positron emission computed tomography-X-ray (PET-X-ray) scanner, a single photon emission computed tomography-magnetic resonance imaging (SPECT-MRI) scanner, a positron emission tomography-computed tomography (PET-CT) scanner, a digital subtraction angiography-magnetic resonance imaging (DSA-MRI) scanner, etc. The medical imaging device 140 provided above is provided for illustrative purposes only and is not intended to limit the scope of the present disclosure.

It should be noted that the application scenario 100 of the system is provided for illustrative purposes only, and is not intended to limit the scope of the present disclosure. For a person of ordinary skill in the art, a variety of modifications or variations may be made according to the description of the present disclosure. As another example, the application scenario 100 may be implemented on other devices to achieve similar or different functions. However, these modifications or variations do not depart from the scope of the present disclosure.

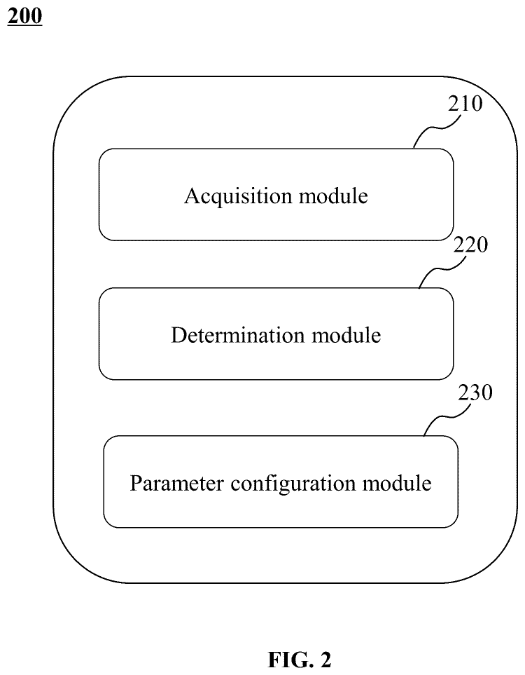

FIG. 2 is a block diagram illustrating an exemplary system for configuring imaging parameters according to some embodiments of the present disclosure. In some embodiments, a system 200 for configuring imaging parameters (referred to as a parameter configuration system 200) may include an acquisition module 210, a determination module 220, and a parameter configuration module 230.

The acquisition module 210 is configured to obtain a user identifier of a user to be examined and one or more device parameters to be set of an imaging device. More descriptions regarding obtaining the user identifier of the user to be examined and one or more device parameters to be set of the imaging device may be found in operation 310 and its related description.

The determination module 220 is configured to determine target values of one or more device parameters based on the user identifier. More descriptions regarding determining target values of one or more device parameters may be found in operation 320 and its related description.

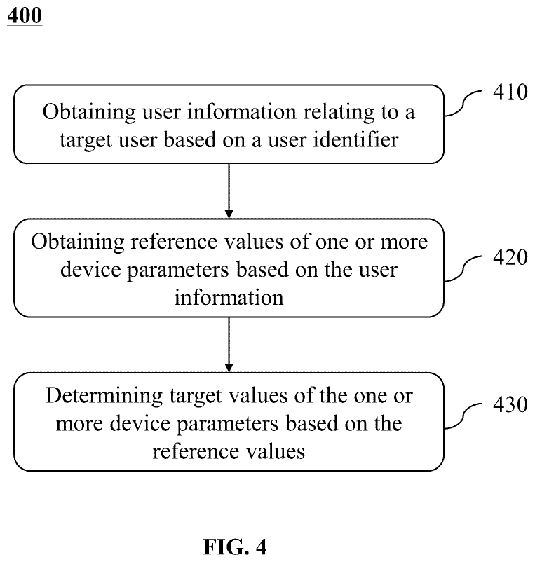

In some embodiments, the determination module 220 is further configured to obtain user information relating to the user to be examined based on the user identifier, obtain reference values of one or more device parameters based on the user information, and determine the target values of one or more device parameters based on the reference value. More descriptions regarding determining the target values of one or more device parameters may be found in FIG. 4 and its related description.

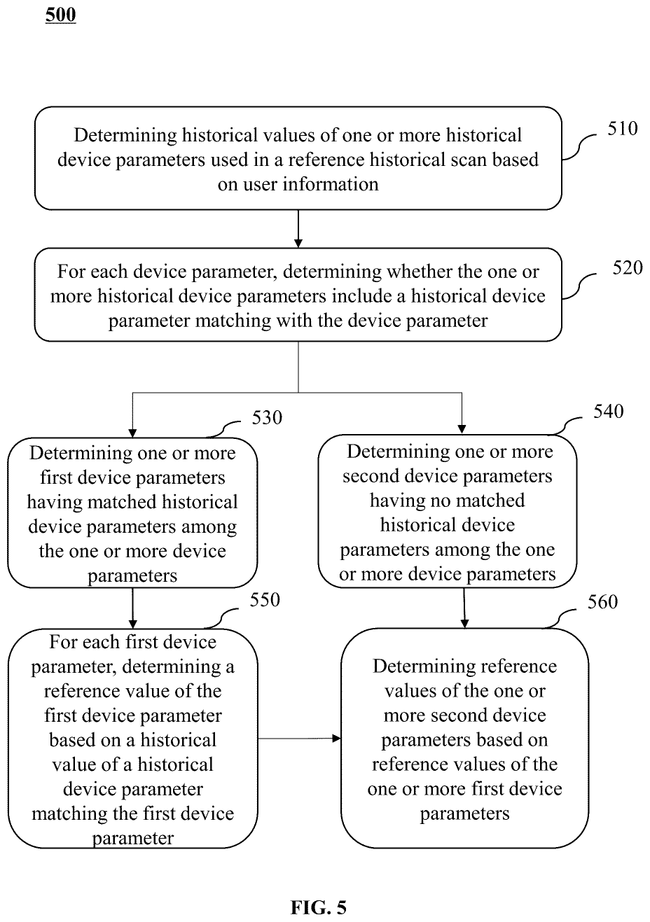

In some embodiments, the determination module 220 is further configured to determine historical values of one or more historical device parameters used in a reference historical scan based on the user information, and determine the reference values of one or more device parameters based on a matching relationship between the one or more historical device parameters and the one or more device parameters. More descriptions regarding determining the reference values of the one or more device parameters may be found in FIG. 5 and its related description.

In some embodiments, the determination module 220 is further configured, for each of the one or more device parameters, to determine whether the one or more historical device parameters include a historical device parameter matching with the device parameter, and in response to determining that the one or more historical device parameters include the historical device parameter matching with the device parameter, determine a reference value of the device parameter based on a historical value of the matched historical device parameter. More descriptions regarding determining the reference value of the device parameter based on the historical value of the matched historical device parameter may be found in operation 520 and its related description.

In some embodiments, the determination module 220 is further configured to determine one or more first device parameters having matched historical device parameters among the one or more device parameters and one or more second device parameters having no matched historical device parameters among the one or more device parameters, and determine reference values of the one or more second device parameters based on reference values of the one or more first device parameters. More descriptions regarding determining the reference values of the one or more second device parameters may be found in operation 520 and its related description.

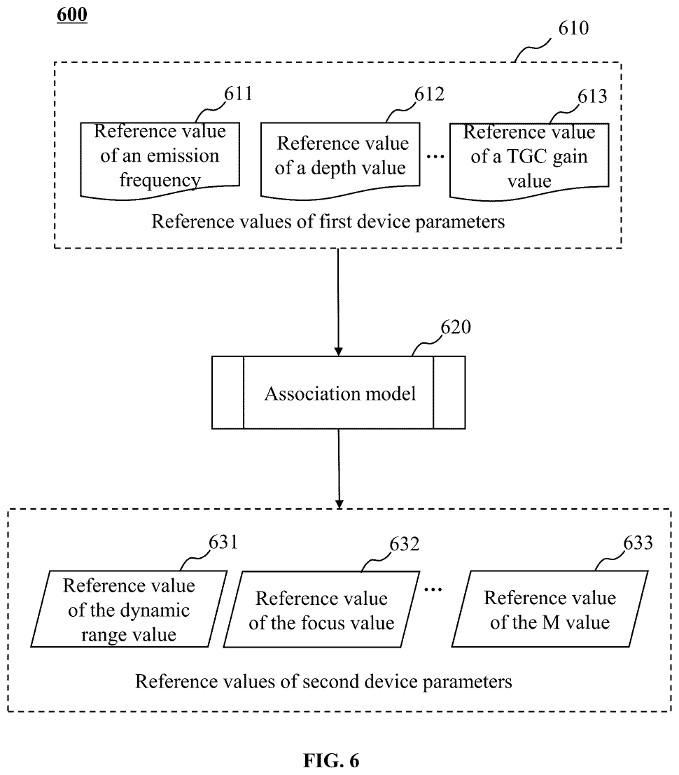

In some embodiments, the determination module 220 is further configured to obtain an association model, the association model reflecting a correlation between the one or more first device parameters and the one or more second device parameters, the association model being a machine learning model. The determination module 220 is further configured to determine the reference values of the one or more second device parameters by processing the reference values of the one or more first device parameters using the association model. More descriptions regarding determining the reference values of the one or more second device parameters using the association model may be found in FIG. 6 and its related description.

In some embodiments, the determination module 220 is further configured to determine a recommendation degree of each of a plurality of sets of reference values, and determine the target values of the one or more device parameters based on the recommendation degree of each set of the plurality of sets of reference values. More descriptions regarding determining the target values of the one or more device parameters based on the recommendation degree of each set of the plurality of sets of reference values may be found in operation 430 and its related description.

In some embodiments, the determination module 220 is further configured to send the plurality of sets of reference values to a user terminal and determine one of the plurality of sets of reference values as the target value based on a selection instruction received from the user terminal. More descriptions regarding determining one of the plurality of sets of reference values as the target value may be found in operation 430 and its related description.

The parameter configuration module 230 is configured to perform parameter configuration on the imaging device based on the target values of the one or more device parameters. More descriptions regarding performing the parameter configuration on the imaging device may be found in operation 330 and its related description.

It is to be noted that the above description of the system and its modules is provided only for descriptive convenience, and does not limit the present disclosure to the scope of the cited embodiments. It is to be understood that for a person skilled in the art, after understanding the principle of the system, it may be possible to arbitrarily combine individual modules or form a subsystem to connect with other modules without departing from this principle. In some embodiments, the acquisition module 210, the determination module 220, and the parameter configuration module 230 disclosed in FIG. 2 may be different modules in a single system, or a single module realizing the aforementioned functions of two or more modules. For example, the individual modules may share a common storage module, and the individual modules may each have a respective storage module. Such morphisms are within the scope of protection of the present disclosure.

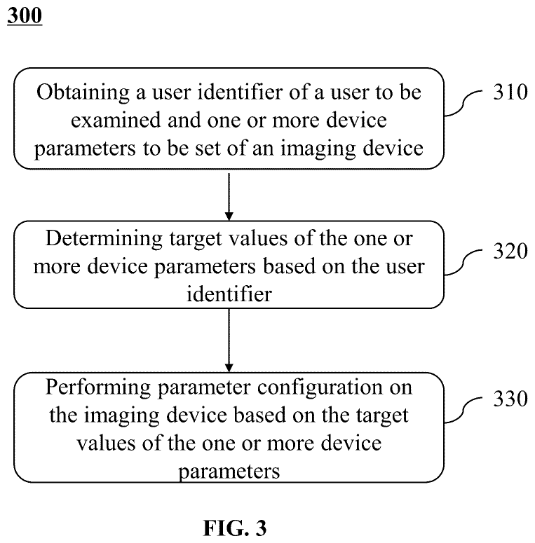

FIG. 3 is a flowchart illustrating an exemplary process for configuring imaging parameters according to some embodiments of the present disclosure. In some embodiments, process 300 may be performed by the parameter configuration system (e.g., the processing device 130 in FIG. 1, one or more modules shown in FIG. 2). As shown in FIG. 3, the process 300 includes the following operations.

In 310, a user identifier of a user to be examined and one or more device parameters to be set of an imaging device are obtained. In some embodiments, the operation 310 may be performed by the acquisition module 210.

The user to be examined refers to a patient who needs to be scanned and examined using the imaging device, such as the medical imaging device 140 described in FIG. 1.

The user identifier refers to a unique identifier that may distinguish different users. In some embodiments, the user identifier of the user to be examined may be used to determine user information relating to the user to be examined. For example, the user information may include identity information of the user to be examined, information relating to a part to be examined, historical examination information, or the like. Detailed descriptions regarding the user information may be found in operation 430 in FIG. 4 and its related description. In some embodiments, the user identifier may be obtained by the acquisition module 210. In some embodiments, the user identifier may include at least one of a numeric identifier and a biometric identifier.

The numeric identifier refers to an identifier generated based on encoding techniques. In some embodiments, the numeric identifier includes at least one of a QR code and a bar code.

To facilitate quick access to the user information of the user to be examined, in some embodiments, the system may attach the numeric identifier to a historical diagnostic report of the user to be examined. For example, after performing a historical scan on the user to be examined using a historical imaging device, the system may encode information such as the identity information, examination information, diagnostic results, or the like, of the user to be examined to generate a QR code and/or a bar code, or other numeric identifiers. The system may add the numeric identifier to the historical diagnostic report when the report is printed or electronically stored. The examination information may include, for example, device information of the historical imaging device, device parameters and parameter values of the device parameters used in the examination of the historical imaging device, image data obtained from the historical scan, or the like.

In some embodiments, the historical imaging device refers to an imaging device that has been used in the historical scan of the user to be examined or a reference user, such as the user to be examined has historically undergone an MRI scan and a CT scan, respectively, an MRI scanner and a CT scanner that perform the MRI scan and the CT scan may be used as the historical imaging device, respectively. The reference user refers to another user that satisfy a predetermined relationship with the user to be examined and have received a historical scan. More descriptions regarding the reference user may be found in operation 410 of FIG. 4 and its related description.

In some embodiments, different numeric identifiers may be utilized to store different information of the user. For example, the QR code may be utilized to store historical examination information of the user to be examined. The bar code may be utilized to store the identity information of the user to be examined, information relating to the part to be examined, or the like.

The biometric identifier refers to an identifier generated from a biometric feature of the user to be examined. In some embodiments, the biometric identifier includes at least one of a fingerprint, a face, a voiceprint, or an iris. In some embodiments, when the user identifier is the biometric identifier, the biometric identifier (e.g., an image, feature information, etc., corresponding to the biometric identifier) of the user and its corresponding user information, etc., may be stored in a storage device (e.g., a cloud server).

In some embodiments, the user identifier includes both the numeric identifier and the biological identifier, which increases the diversity of the user identifier and improves the convenience of the user to be examined. At the same time, because the biological identifier has the property of not being easily lost or damaged, it may effectively prevent the failure to obtain the user information due to the loss or damage of the user identifier when the user receives the follow-up examination.

In some embodiments, the acquisition module 210 may obtain different forms of user identifiers in different ways.

In some embodiments, the numeric identifier may be obtained from a paper or electronic historical diagnostic report of the user to be examined. For example, the QR code from the paper historical diagnostic report may be detected by a detection device (e.g., the detection device 120) as the user identifier for the user to be examined. The user identifier obtained by the detection device may be sent to the acquisition module 210 for further analysis. As another example, the acquisition module 210 may perform image or text recognition of the electronic historical diagnostic report to obtain a bar code thereof as the user identifier of the user to be examined.

In some embodiments, the biometric identifier may be obtained by scanning or detecting the biometric feature of the user to be examined. For example, the detection device (e.g., detection device 120) may be utilized to scan facial features and fingerprint features of the user to be examined, or perform voice recognition on the user to obtain the biometric identifier of the user.

The imaging device refers to a medical device used to perform a scan and an examination on the user to be examined. For example, the imaging device may be medical imaging devices such as a magnetic resonance (MR) device, a computed tomography (CT) detection device, or the like. Detailed descriptions regarding the medical imaging device may be found in FIG. 1 and its related description.

The device parameters refer to parameters that need to be set before the imaging device scans the user to be examined. For example, before scanning the user to be examined using the MRI device, the device parameters that need to be set may include an examination mode, an emission frequency, a depth value, a time gain compensate (TGC), a dynamic range value, a focal point value, and an M value. As another example, when X-ray scanning of the human body is performed using the CT device, the device parameters that need to be set up may include a bulb tube current, a scanning duration, a scanning dose, etc.

In some embodiments, one or more device parameters of the imaging device may be obtained by the acquisition module 210. In some embodiments, the one or more device parameters to be set of the imaging device may be obtained based on a variety of ways. For example, the one or more device parameters to be set of the imaging device may be determined based on parameters that need to be set in an operator interface of the imaging device. As another example, the one or more device parameters to be set of the imaging device may be obtained based on information such as a model number of the imaging device and a part to be scanned of the user to be examined.

In some embodiments, when the user to be examined goes to a hospital for a follow-up examination, the detection device may directly scan the numeric identifier on the historical diagnostic report held by the user to be examined. Based on the scanning of the numeric identifier by the detection device, the acquisition module 210 may obtain the user information of the user to be examined to quickly determine the device parameters of the current scan based on the user information. In some embodiments, the user information may include historical device parameters of a historical imaging device that performs the historical scan of the user. When the model number, manufacturer, etc. of the historical imaging device is identical to the model number, manufacturer, etc. of the imaging device used in the current follow-up examination of the user to be examined, the acquisition module 210 may determine the device parameters of the imaging device in the current follow-up examination based on the historical device parameters.

In 320, target values of the one or more device parameters are determined based on the user identifier. In some embodiments, operation 320 may be performed by the determination module 220.

The target values of the device parameters refer to parameter values that the imaging device may use in the scan of the user to be examined. For example, the device parameters to be set of the imaging device include an examination mode, an emission frequency, a depth value, a TGC gain value, a dynamic range value, a focal point value, and an M value, etc. The target value of the examination mode is a, the target value of the emission frequency is b, the target value of the depth value is c, the target value of the TGC gain value is d, the target value of the dynamic range value is e, the target value of the focal point value is f, and the target value of the M value is g. The parameters of the imaging device may be set based on the target values, and then the imaging device may be utilized to perform a scan on the user to be examined to obtain scan data.

In some embodiments, the system may obtain the target values of the one or more device parameters based on the user identifier in a plurality of ways. For example, the system may preset device parameters and target values of the device parameters corresponding to various types of users to be examined (e.g., users to be examined categorized according to information such as an age, a gender, a medical condition, and a scanning part). After obtaining the user information by scanning the user identifier of the user to be examined, a category that the user to be examined belongs may be determined and the target values of the device parameters may be determined based on the preset information.

In some embodiments, the system may determine the reference values of the one or more device parameters based on the user information, and then determine the target values based on the reference values. More descriptions regarding determining the target values based on the reference values may be found in FIG. 4 and its related description.

In 330, parameter configuration is performed on the imaging device based on the target values of the one or more device parameters. In some embodiments, operation 330 may be performed by the parameter configuration module 230.

In some embodiments, the parameter configuration module 230 may directly use the target values of the device parameters as the parameter values of the imaging device. In some embodiments, the parameter configuration module 230 may also take target values of device parameters modified by the user as the parameter values of the imaging device. For example, the user may modify a portion of the target values of the device parameters based on his or her own experience, and the parameter configuration module 230 may use the modified and unmodified target values as the parameter values of the imaging device.

In some embodiments of the present disclosure, the user's identity and his or her historical diagnostic information, diagnostic results, etc., may be quickly determined by the user identifier, and the target values of the device parameters of the current scan may be quickly determined based on the historical device parameters used in the historical scan in the historical diagnosis, thereby realizing the parameter configuration on the imaging device based on the target values of the device parameters. Therefore, there is no need for the user of the imaging device to re-enter the user information of the user to be examined and the parameter values of the imaging device is quickly obtained, which makes it easier for the user to be examined to go to the hospital for a follow-up examination, and shortens the process and time of the scanning and imaging process during the follow-up examination.

FIG. 4 is a flowchart illustrating an exemplary process for determining target values of device parameters according to some embodiments of the present disclosure. In some embodiments, process 400 may be performed by the determination module 220. In some embodiments, operation 320 in process 300 may be realized by the process 400. As shown in FIG. 4, the process 400 includes the following operations.

In 410, user information relating to the user is obtained based on a user identifier.

In some embodiments, the user information may include relevant information about the user to be examined. The relevant information may include personal information and historical examination information. For example, the user information may include the identity information (e.g., a name, an age, a gender, a cell phone number, an identity card number, a hospitalization number) of the user to be examined or a reference user, information of a part to be examined of the user to be examined, historical scan images captured by the historical scans of the user to be examined, basic device information and probe information (e.g., a model number of the probe) of the historical imaging devices that perform the historical scans on the user to be examined, and historical device parameters and parameter values of the historical device parameters that used in the historical scans.

In some embodiments, the user information may include information relating to one or more reference users corresponding to the user to be examined. A reference user refers to a user that satisfies a predetermined relationship with the user to be examined and has received a historical scan. The predetermined relationship may be a relationship that needs to be satisfied between the user information (or referred to as user features) of the user to be examined and reference user information (or referred to as reference user features) of the reference user. The predetermined relationship may be that the ages fall within the same age range, the genders are the same, the scanning parts are the same or similar, etc. Merely by way of example, if the current scan of the user to be examined is an MRI scan, the reference user may be another user whose age and gender are the same as that of the user to be examined, who has previously undergone an MRI scan and whose scan part and symptoms are similar or the same as those of the user to be examined. In some embodiments, relevant information of the reference user may be determined based on the identity information and historical examination information of the user.

In some embodiments, the numeric identifier on the historical diagnostic report may be scanned with the detection device to obtain a storage address (e.g., a corresponding URL address) that stores the user information of the user to be examined and is documented within the numeric identifier, and the determination module 220 may read the user information of the user to be examined based on the storage address.

In some embodiments, a biometric feature of the user to be examined may be scanned by the detection device, and the biometric identifier may be sent to the determination module 220. The determination module 220 may determine the user identity corresponding to the biometric feature and the storage address of the corresponding user information after matching with the biometric feature with a plurality of sets of stored biometric features. The determination module 220 reads the user information of the user to be examined according to the storage address.

In 420, reference values of one or more device parameters are obtained based on the user information.

The reference values refer to recommended values of the device parameters to be set of the imaging device.

In some embodiments, the determination module 220 may determine, based on the user information, one or more reference historical scans that the user to be examined and/or the one or more reference users have received. The determination module 220 may further determine the reference values of the device parameters of the current scan based on historical values of one or more historical device parameters used in the one or more reference historical scans.

A reference historical scan refers to a historical scan that is the same or similar to the current scan among historical scans of the user to be examined or the reference user. For example, if the current examination of the user to be examined is an MRI of the chest, the reference historical scan may include an MRI of the chest that has been performed on the user to be examined or the reference user. As another example, if the current examination of the user to be examined is a CT scan of the left leg, the reference historical scan may include the CT scan of the left leg or the right leg that has been performed on the user to be examined or the reference user. More descriptions regarding determining the reference values of the device parameters of the current scan based on the historical values of the historical device parameters may be found in FIG. 5 and its related description.

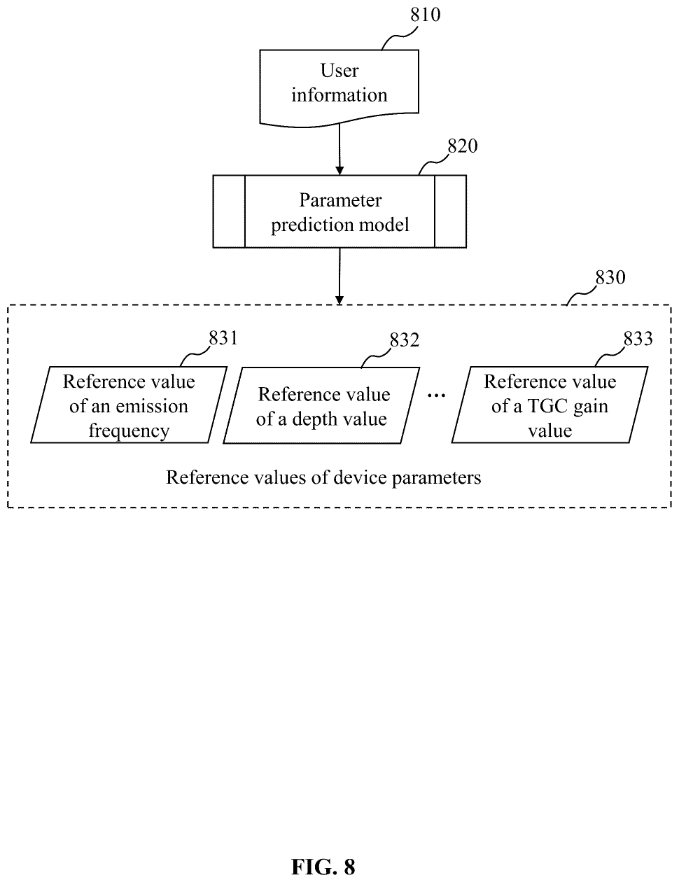

In some embodiments, the determination module 220 may process the user information based on a parameter prediction model to obtain the one or more reference values of the device parameters. More descriptions regarding determining the reference values of the device parameters based on the parameter prediction model may be found in FIG. 8 and its related description.

In some embodiments, different device parameters may be determined in the same way, or in different ways. For example, some device parameters may be determined based on the historical values of the historical device parameters, and other device parameters may be determined based on the parameter prediction model.

In 430, target values of the one or more device parameters are determined based on the reference values.

In some embodiments, the determination module 220 may determine the target values of the one or more device parameters based on the reference values in a plurality of ways. For example, the determination module 220 may directly determine a reference value of a device parameter as the target value of the device parameter. As another example, the determination module 220 may use a reference value of a device parameter modified by the user (e.g., a doctor) as the target value of the device parameter.

In some embodiments, the reference values of the device parameters may include one or more sets of reference values. For example, the reference values of the device parameters may include a reference value set A, a reference value set B, and a reference value set C. The reference value set A is determined based on a historical scan of the user to be examined, the reference value set B is determined based on a historical scan of the reference user, and the reference value set C is determined based on the parameter prediction model. When the reference values of the device parameters include a plurality of sets of reference values, the determination module 220 may first determine a recommendation degree of each set of the plurality of sets of reference values, and then determine, based on the recommendation degree of each set of the plurality of sets of reference values, the target values of the device parameters. For example, the determination module 220 may use the set of reference values with the highest recommendation degree as the target values of the device parameters. As another example, the determination module 220 may determine three sets of reference values with the highest recommendation degree, and use an average of the three sets of reference values as the target values of the device parameters.

The recommendation degree of a set of reference values refers to a recommendation degree to use the set of reference values as the target values of the imaging device of the user to be examined in the current examination. The recommendation degree may be expressed as a real number between 0 and 1, and the larger the value, the higher the recommendation degree. For example, the recommendation degrees may be 0.1 or 0.5, and a set of reference values with a recommendation degree of 0.5 is more suitable for use as the target values of the imaging device in the current examination compared to another set of reference values with a recommendation degree of 0.1.

In some embodiments, the plurality of sets of reference values may be reference values determined based on different ways. For example, the plurality of sets of reference values may include a first set of reference values and a second set of reference values. The first set of reference values refers to reference values of the device parameters determined based on a reference historical scan. For example, the first set of reference values may be determined based on the historical values of one or more historical device parameters used in the reference historical scan.

In some embodiments, the recommendation degree of the first set of reference values may be determined based on a matching relationship between the one or more historical device parameters of the reference historical scan and the one or more device parameters of the current scan. The matching relationship refers to a similarity relationship between the historical device parameters of the reference historical scan and the one or more device parameters to be set among the device parameters of the current scan. For example, if a device parameter to be set of the current scan is included in the historical device parameters of the reference historical scan, it is assumed that the device parameter to be set matches with the corresponding historical device parameter, otherwise, the device parameter to be set is considered to be mismatched with the corresponding historical device parameter. Merely by way of example, if a device parameter to be set in a current CT scan includes a bulb tube current, and a value of the bulb tube current is also set in a reference historical CT scan, it is considered that the bulb tube current to be set in the current scan matches with the bulb tube current in the reference historical CT scan.

In some embodiments, the recommendation degree of the first set of reference values may be determined based on a count of device parameters that match with the historical device parameters among the device parameters of the current scan. For example, if the historical device parameters contain all of the device parameters to be set in the current scan of the imaging device, the recommendation degree of the first set of reference values may be a larger value, such as a recommendation degree of 1. As another example, if the historical device parameters contain half of the device parameters to be set in the current scan, the recommendation degree of the first set of reference values may be 0.5.

The second set of reference values refers to parameter values of the device parameters determined based on the parameter prediction model. For example, the determination module 220 may process the user information using the parameter prediction model to determine the parameter values of the device parameters and use the parameter values determined by the parameter prediction model as the second set of reference values of the device parameters.

In some embodiments, the recommendation degree of the second set of reference values may be determined based on a count of usage of the second set of reference values by target reference users in target devices. A target reference user refers to a reference user having the same age, gender, examination part, scanning item, or the like, as the user to be examined. A target device refers to a device of the same type (e.g., the same model number) as the imaging device used by the user to be examined in the current examination scan. In some embodiments, the reference user may be directly designated as the target reference user. For example, if the second set of references is used 3 times in 5 scans by the target device on 5 different target reference users or the same target reference user in a certain historical time period, the recommendation degree of the second set of references may be 0.6.

In some embodiments, the determination module 220 may compare the recommendation degree of the first set of reference values and the recommendation degree of the second set of reference values, and select the set of reference values with the greatest recommendation degree as the target values of the one or more device parameters.

According to some embodiments of the present disclosure, the determination module 220 determines the target values of the one or more device parameters by comparing the recommendation degree of each set of the plurality of sets of reference values, which makes the final target values more reasonable, thereby effectively improving the scanning imaging quality of the imaging device.

In some embodiments, when the reference values of the device parameters include a plurality of sets of reference values, the determination module 220 may send the plurality of sets of reference values to the user terminal, and then determine one set of the plurality of sets of reference values as the target values based on a selection instruction received from the user terminal.

The user terminal refers to a terminal that interacts with the user of the imaging device. For example, the user terminal may be an operator panel on an MRI machine. Alternatively, the user terminal may be a tablet in wireless communication with the MRI machine.

The selection instruction refers to a control instruction input by the user via the user terminal for selecting one set of reference values from the plurality of sets of reference values. For example, the user may input the selection instruction by voice, gesture, typing, etc. In some embodiments, if the user does not enter the selection instruction within the predetermined time period, the determination module 220 defaults to using the parameter values of the device parameters determined based on the parameter prediction model as the target values. The predetermined time period may be set artificially in advance. For example, the predetermined time period may be one minute. In some embodiments, the user may also compare the plurality of sets of reference values to modify his or her selected reference values. The modified reference values may be used as the target values.

According to some embodiments of the present disclosure, the target values of the one or more device parameters are determined by sending the plurality of sets of reference values to the user terminal for selection by the user of the imaging device. The method for configuring imaging parameters disclosed in the present disclosure is automated and may reduce the workload of the user of the imaging device as compared to directly having the user enter the target values and may improve the efficiency and accuracy of the device parameters of the imaging device (e.g., avoiding setting errors due to subjective differences in the user).

According to some embodiments of the present disclosure, the user information may be obtained using the user identifier, and then the reference values may be obtained based on the user information, and ultimately the target values of the one or more devices may be determined, which is not only conducive to simplifying the process of configuring the parameters for the user to be examined and shortening the follow-up examination time when the user receives the follow-up examination, but also makes the parameter settings of the imaging device more reasonable to improve the imaging quality of imaging device.

It should be noted that the foregoing descriptions of the process 300, the process 400 are for the purpose of exemplification and illustration only, and do not limit the scope of application of the present disclosure. For a person skilled in the art, various corrections and changes can be made to the process 300, the process 400 under the guidance of the present disclosure. However, these corrections and changes remain within the scope of the present disclosure. For example, operation 420 may be omitted. The determination module 220 may determine the target values of one or more device parameters based directly on the user identifier.

FIG. 5 is a flowchart illustrating an exemplary process for determining reference values of device parameters according to some embodiments of the present disclosure. In some embodiments, process 500 may be performed by the determination module 220. In some embodiments, operation 420 in the process 400 may be realized by the process 500. As shown in FIG. 5, the process 500 includes the following operations.

In 510, historical values of one or more historical device parameters used in a reference historical scan is determined based on user information.

The reference historical scan refers to a historical scan that is the same or similar to the current scan of a user to be examined or a reference user. More descriptions regarding the reference historical scan may be found in operation 430 in FIG. 4 and its related description.

In some embodiments, the determination module 220 may determine the reference historical scan by the following operations. First, the determination module 220 may select a target reference user from at least two reference users based on the user information. Each of the at least two reference users receives a historical scan performed by a historical imaging device. The determination module 220 may then determine the historical scan of the target reference user as the reference historical scan. More descriptions regarding the reference user may be found in operation 410 in FIG. 4 and its related description.

The target reference user refers to a reference user that is scanned and imaged using an imaging device with the same or similar model number as the user to be examined, and whose user information has a similarity not less than a similarity threshold with the user information of the user to be examined. The user information of the reference user is referred to as the reference user information hereinafter. The similarity threshold may be set in advance. For example, when the similarity threshold is 100% and the user to be examined in the current examination is scanned using an MRI scanner, the target reference user may be a user who has the same age, gender, medical condition, examination part, etc., as the user to be examined, and uses an MRI scanner with the same or similar model number as the user to be examined for the scanning.

In some embodiments, the determination module 220 may directly use a reference user who has the same age, weight, medical condition, examination part, etc., as the user to be examined and uses the imaging device with the same model number as the user to be examined for the scanning as the target reference user. More detailed descriptions regarding the target reference user may be found in FIG. 4 and its related description.

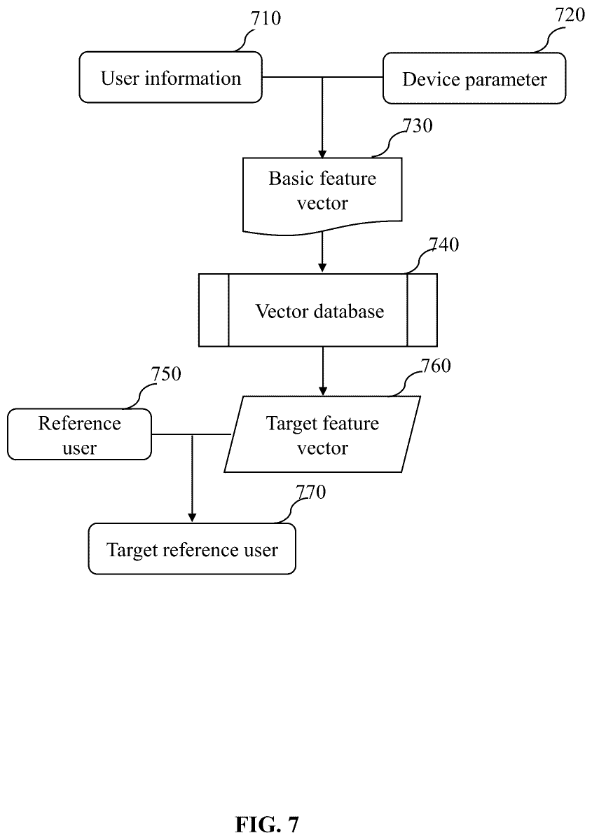

In some embodiments, the determination module 220 may also determine the target reference user based on a vector database and a basic feature vector. More descriptions regarding determining the target reference user may be found in FIG. 7 and its related description.

In some embodiments, after determining the target reference user, the determination module 220 may directly designate the historical scan of the target reference user as the reference historical scan of the user to be examined.

In some embodiments, the determination module 220 may also obtain, based on the user information, a historical scan of the user to be examined performed by a reference imaging device, and designate the historical scan of the user to be examined as the reference historical scan.

The reference imaging device refers to another imaging device that has performed the historical scan on the user to be examined and has the same or similar model number as the imaging device used in the current scan. For example, for an MRI scanner used in the current scan of the user to be examined, and the reference imaging device may include another MRI scanner that has performed the historical scan on the user to be examined. The determination module 220 may obtain, based on the user information, historical scans of the current user to be examined performed by the reference imaging device and corresponding scan results and the parameter settings of the reference imaging device. The determination module 220 may designate, among the historical scans, the historical scan that has the same scanning part as the current scan as the reference historical scan and perform parameter configuration on the imaging device in the current scan based on the parameter values of the device parameters of the reference imaging device in the reference historical scan.

By determining the parameters of the current imaging device based on the device parameters of the scan of the same user to be examined performed by other imaging devices as a reference, fast and efficient completion of the parameter configuration of the imaging device may be realized, thus improving the efficiency of utilizing the medical imaging device to examine patients.

In some embodiments, the determination module 220 may adjust the parameter values of the device parameters of the reference imaging device according to a performance index of the imaging device of the current scan and a performance index of the reference imaging device corresponding to the reference historical scan, to perform the parameter configuration on the imaging device in the current scan. For example, if the imaging device used in an initial examination of the user to be examined requires a dose a of radiation to obtain an image with a uniform resolution and the current imaging device requires only a dose b of radiation, the determination module 220 may determine the parameter configuration on the imaging device in the current scan utilizing a relationship that a ratio of the parameter values of the device parameters of the reference imaging device to the dose a is equal to a ratio of the parameter values of the device parameters of the imaging device in the current scan to the dose b.

In some embodiments of the present disclosure, the reference historical scan is obtained by selecting the reference users based on the user information and determining the target reference user, which makes the reference historical scan more informative and may effectively improve the applicability of the parameter values used in the reference historical scan to the user to be examined.

The historical device parameters refer to device parameters used in the reference historical scan performed by the historical imaging device. In some embodiments, the historical imaging device may be of the same type as the imaging device. In some embodiments, the historical device parameters may include one or more device parameters (or a portion thereof) that need to be set in the current scan or may include one or more other device parameters.

In some embodiments, after determining the reference historical scan based on the user information, the determination module 220 may obtain historical values of one or more historical device parameters used in the reference historical scan. For example, the determination module 220 may obtain identity information of the user based on a numeric identifier or a biometric identifier, determine the reference historical scan based on the identity information of the user and further obtain historical values of the one or more historical device parameters used in the reference historical scan.

The count of the reference historical scan determined in operation 510 may be one or more. For illustrative purposes, the following describes how to determine reference values of one or more device parameters based on the historical values of one or more historical device parameters used in a single reference historical scan.

In some embodiments, the determination module 220 may determine the reference values of the one or more device parameters based on a matching relationship between the one or more historical device parameters and the one or more device parameters. The matching relationship may indicate whether the historical device parameters contain historical device parameters that match with the parameters to be set. If a device parameter and a historical device parameter belong to the same parameter type, they are considered to match each other. For example, if the historical device parameters contain an emission frequency, and the current device parameters to be set also contain the emission frequency, the emission frequency to be set is considered to have a historical device parameter matching with the emission frequency.

In some embodiments, for each device parameter, the determination module 220 may determine a reference value corresponding to the device parameter. For example, for each of the one or more device parameters, the determination module 220 may determine whether the one or more historical device parameters include a historical device parameter matching with the device parameter. In response to determining that the one or more historical device parameters include a historical device parameter matching with the device parameter, the determination module 220 may determine a reference value of the device parameter based on a historical value of the matched historical device parameter. For example, when the one or more historical device parameters includes the historical device parameter matching with the device parameter, the determination module 220 may directly designate the historical value of the matched historical device parameter as the reference value of the device parameter. Based on the historical value of the matched historical device parameter, the reference value of the device parameter may be quickly determined, thereby improving the accuracy and efficiency of parameter configuration.

In some embodiments, one or more of operations 520-560 may be performed to determine the reference value based on the matching relationship.

In 520, for each of the one or more device parameters, whether the one or more historical device parameters include the historical device parameter matching with the device parameter is determined.

In 530, one or more first device parameters having matched historical device parameters among the one or more device parameters are determined.

In 540, one or more second device parameters having no matched historical device parameters among the one or more device parameters are determined.

For example, the historical device parameters include an examination mode, an emission frequency, a depth value, a TGC gain value. The examination mode, the emission frequency, and the depth value belong to the first device parameters, and the TGC gain value belongs to the second device parameters.

If one or more first device parameters exist, operation 550 may be performed. In 550, for each of the one or more first device parameters, a reference value of the first device parameter is determined based on a historical value of a historical device parameter matching with the first device parameter. For example, the historical value of the historical device parameter matching with the first device parameter may be determined as the reference value of the first device parameter.

If one or more second device parameters exist, operation 560 may be performed. In 560, reference values of the one or more second device parameters may be determined based on reference values of the one or more first device parameters.

In some embodiments, the determination module 220 may determine the reference values of the one or more second device parameters based on the reference values of the one or more first device parameters in a plurality of ways. For example, the reference values of the second device parameters may be determined empirically by the user of the imaging device based on the reference values of the first device parameters, in combination with other information of the user to be examined (e.g., the age, gender, condition, and examination part of the patient to be examined).

In some embodiments, the determination module 220 may process the reference values of the first device parameters utilizing an association model to determine the reference values of the second device parameters. The association model may reflect a correlation between the first device parameters and the second device parameters. In some embodiments, the association model is a machine learning model. For example, the association models may include a neural network (NN) model, a convolutional neural network (CNN) model, a recurrent neural network (RNN) model, etc.

For exemplary purposes, FIG. 6 is a schematic diagram illustrating an association model according to some embodiments of the present disclosure. As illustrated in FIG. 6, the determination module 220 may input reference values 610 of the one or more first device parameters into an association model 620, and the association model 620 may output reference values 630 of the one or more second device parameters. Merely by way of example, the device parameters include an emission frequency, a depth value, a TGC gain value, a dynamic range value, a focus value, and an M value. The emission frequency, the depth value, and the TGC gain value may match with historical device parameters in a reference historical scan, the emission frequency, the depth value, and the TGC gain value may be the first device parameters. The dynamic range value, the focus value, and the M value may not match with the historical device parameters in the reference historical scan, then the dynamic range value, the focus value, and the M value may be the second device parameters. At this time, a reference value of the emission frequency 611, a reference value of the depth value 612, and a reference value of the TGC gain value 613 may be input into the association model. The association model 620 may output a reference value of the dynamic range value 631, a reference value of the focus value 632, and a reference value of the M value 633.

In some embodiments, the association model 620 may be obtained by training a plurality of first training samples with labels. For example, each of the plurality of first training samples with labels may be input into an initial association model, a value of a loss function is calculated from the label and an output result of the initial association model, and the initial association model is iteratively updated based on the value of the loss function. When the initial association model satisfies a predetermined condition, the model training is completed, and the trained association model is obtained. The predetermined condition may be that the loss function converges, a count of iterations reaches a threshold, etc.

In some embodiments, the first training samples may include first sample values of one or more first device parameters, and the labels may be second sample values corresponding to one or more second device parameters. In some embodiments, the labels may be obtained by manual labeling.

In some embodiments, the first training samples and the labels may be obtained based on a plurality of ways. For example, historical values of all historical device parameters may first be obtained from a historical scanning record, one or more historical device parameters may be randomly deleted from the historical device parameters, and historical values of the remaining historical device parameters may be designated as the first training sample, and historical values of the one or more deleted historical device parameters may be designated as labels. As another example, historical values of historical device parameters matching with the first device parameters may be designated as the first training samples, and historical values of historical device parameters matching with the second device parameters may be designated as the labels.

In some embodiments, the labels may also be a relationship (e.g., a ratio, a difference, a multiplier, etc.) between the first sample values and the second sample values, etc. At this point, when the reference values of the first device parameters are processed using the association model, the association model may output a predicted relationship between the reference values of the first device parameters and the reference values of the second device parameters. The determination module 220 may determine the reference values of the second device parameters based on the reference values of the first device parameters and the predicted relationship.

In some embodiments of the present disclosure, by processing of the reference values of the first device parameters using the association model, the reference values of the second device parameters may be determined based on a correlation between the second device parameters and other parameters values. Therefore, the reference values of the second device parameters may be more reasonable and accurate, which is conducive to improving the quality of subsequent scans of the imaging device.

In some embodiments of the present disclosure, the reference values of the device parameters may be determined in the case of insufficient historical device parameters by determining the reference values of the second device parameters through the reference values of the first device parameters utilizing a model or human experience, thus improving the efficiency of parameter setting.

It should be noted that the foregoing description of the process 500 is intended to be exemplary and illustrative only and does not limit the scope of application of the present disclosure. For a person skilled in the art, various corrections and changes may be made to the process 500 under the guidance of the present disclosure. However, these corrections and changes remain within the scope of the present disclosure. For example, operation 550, the reference values of the one or more first device parameters may also be determined based on the reference values of the other first device parameters in the manner of operation 560. As another example, the reference values of the one or more second device parameters may be artificially set by the user.

FIG. 7 is a flowchart illustrating an exemplary process for determining a target reference user according to some embodiments of the present disclosure. In some embodiments, operation 510 in process 500 may be accomplished by a flow as shown in FIG. 7.

As mentioned above, the target reference user refers to a reference user whose user information has a similarity not less than a similarity threshold with the user information of the user to be examined. More descriptions regarding the target reference user may be found in operation 510 and its related description.

In some embodiments, the determination module 220 may construct a basic feature vector 730 corresponding to the user to be examined based on the user information 710 of the user to be examined and device information 720 relating to the imaging device that may scan the user to be examined. More descriptions regarding the user information may be found in operation 410 and its related description. The device information 720 refers to information relating to the imaging device. For example, the device information 720 may include information such as a type, a model number, and other information of the imaging device. The basic feature vector 730 refers to a vector for storing feature values included in the user information 710 and the device information 720. The vector database 740 includes reference feature vectors of a plurality of reference users. A reference user may be a user that has undergone a historical scan performed by a historical reference device. The reference feature vectors include feature values contained in the user information of the reference users and the device information of the historical imaging devices. The feature values in the basic feature vector 730 and the reference feature vector correspond to the same features.

In some embodiments, the determination module 220 may obtain a target feature vector 760 in the vector database 740 based on the basic feature vector 730 and designate a reference user 750 corresponding to the target feature vector 760 as a target reference user 770.

In some embodiments, the target feature vector 760 may be determined based on a similarity between the basic feature vector 730 and the reference feature vectors of the plurality of reference users in the vector database 740. For example, a reference feature vector whose similarity with the basic feature vector 730 satisfies a predetermined similarity condition may be designated as the target feature vector 760. The predetermined similarity condition may be set according to the situation. For example, the similarity is maximum, or the similarity is greater than a threshold, etc. Similarity between vectors may be measured by a vector distance, which may include, but is not limited to, a Euclidean distance, a cosine distance, a Mahalanobis distance, a Chebyshev distance, and/or a Manhattan distance, etc.

In some embodiments of the present disclosure, by determining the target feature vector in the vector database based on the basic feature vector to determine the target reference user, a user with information (e.g., the age, medical condition, scanning part, etc.,) that is closest to the information of the user to be examined may be selected as the target reference user. This enables the parameter values of the device parameters of the target reference user to be more appropriate for the scanning of the user to be examined, which is conducive to the improving the accuracy and efficiency of parameter setting.

FIG. 8 is a schematic diagram illustrating a parameter prediction model according to some embodiments of the present disclosure. In some embodiments, operation 420 in process 400 may be realized by a parameter prediction model as shown in FIG. 8.

In some embodiments, the determination module 220 may determine at least a portion of reference values 830 of one or more device parameters by processing user information 810 using a parameter prediction model 820. The parameter prediction model 820 is a machine learning model.

In some embodiments, the parameter prediction model 820 may include a neural network (NN) model, a convolutional neural network (CNN) model, a recurrent neural network (RNN) model, or the like.

In some embodiments, an input of the parameter prediction model 820 may include the user information 810. More descriptions regarding the user information may be found in operation 410 and its related description. An output of the parameter prediction model 820 may include reference values 830 of the one or more device parameters. For example, the reference values 830 of the one or more device parameters may include a reference value of an emission frequency 831, a reference value of a depth value 832, a reference value of a TGC gain value 833, or the like.

In some embodiments, the parameter prediction model 820 may be obtained by training a plurality of second training samples with labels. For example, each of the plurality of second training samples may be input into an initial parameter prediction model, a value of a loss function is calculated from the label and an output result of the initial parameter prediction model, and parameters of the initial parameter prediction model are iteratively updated based on the value of the loss function. When the initial parameter prediction model satisfies a predetermined condition, the model training is completed, and the trained parameter prediction model is obtained. The predetermined condition may be that the loss function converges, a count of iterations reaches a threshold, etc.

In some embodiments, a second training sample may be user information of a user in a historical scan, and the label may be parameter values of historical device parameters corresponding to the user information. The training samples and the labels may be obtained from a historical scan database.

In some embodiments, the determination module 220 may train different parameter prediction models for different types of imaging devices. When the user to be examined needs to be scanned, the corresponding parameter prediction model may be selected based on the type of imaging device used for the scanning to obtain the reference values of the device parameters. For example, two sets of training samples and labels may be prepared based on a CT scanner and a MRI scanner, respectively, and are used for training a parameter prediction model A for device parameter prediction of the CT scanner and a parameter prediction model B for device parameter prediction of the MRI scanner, respectively. If a CT scan needs to be performed on the current user to be examined, the parameter values may be predicted by the parameter prediction model A.

In some embodiments of the present disclosure, predicting the reference values of the one or more device parameters by the parameter prediction model may improve the accuracy of the reference values of the device parameters, and make the parameter values of the device parameters more reasonable for the user to be examined.

Having thus described the basic concepts, it may be rather apparent to those skilled in the art after reading this detailed disclosure that the foregoing detailed disclosure is intended to be presented by way of example only and is not limiting. Although not explicitly stated here, those skilled in the art may make various modifications, improvements, and amendments to the present disclosure. These alterations, improvements, and amendments are intended to be suggested by this disclosure and are within the spirit and scope of the exemplary embodiments of the present disclosure.

Moreover, certain terminology has been used to describe embodiments of the present disclosure. For example, the terms “one embodiment,” “an embodiment,” and/or “some embodiments” mean that a particular feature, structure, or feature described in connection with the embodiment is included in at least one embodiment of the present disclosure. Therefore, it is emphasized and should be appreciated that two or more references to “an embodiment” or “one embodiment” or “an alternative embodiment” in various portions of the present disclosure are not necessarily all referring to the same embodiment. In addition, some features, structures, or characteristics of one or more embodiments in the present disclosure may be properly combined.

Furthermore, the recited order of processing elements or sequences, or the use of numbers, letters, or other designations, therefore, is not intended to limit the claimed processes and methods to any order except as may be specified in the claims. Although the above disclosure discusses some embodiments of the invention currently considered useful by various examples, it should be understood that such details are for illustrative purposes only, and the additional claims are not limited to the disclosed embodiments. Instead, the claims are intended to cover all combinations of corrections and equivalents consistent with the substance and scope of the embodiments of the present disclosure. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that object of the present disclosure requires more features than the features mentioned in the claims. Rather, claimed subject matter may lie in less than all features of a single foregoing disclosed embodiment.

In some embodiments, the numbers expressing quantities or properties used to describe and claim certain embodiments of the present disclosure are to be understood as being modified in some instances by the term “about,” “approximate,” or “substantially.” For example, “about,” “approximate” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes. History application documents that are inconsistent or conflictive with the contents of the present disclosure are excluded, as well as documents (currently or subsequently appended to the present specification) limiting the broadest scope of the claims of the present disclosure. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the present disclosure disclosed herein are illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. A method for configuring imaging parameters, implemented on a computing device having at least one processor and at least one storage device, comprising:
obtaining a user identifier of a user to be examined and one or more device parameters to be set of an imaging device;
determining target values of the one or more device parameters based on the user identifier; and
performing parameter configuration on the imaging device based on the target values of the one or more device parameters.

2. The method of claim 1, wherein the determining target values of the one or more device parameters based on the user identifier includes:
obtaining user information relating to the user based on the user identifier;
obtaining reference values of the one or more device parameters based on the user information; and
determining the target values of the one or more device parameters based on the reference values.

3. The method of claim 2, wherein the obtaining reference values of the one or more device parameters based on the user information includes:
determining historical values of one or more historical device parameters used in a reference historical scan based on the user information; and
determining the reference values of the one or more device parameters based on a matching relationship between the one or more historical device parameters and the one or more device parameters.

4. The method of claim 3, wherein the determining the reference values of the one or more device parameters based on a matching relationship between the one or more historical device parameters and the one or more device parameters includes:
for each of the one or more device parameters,
determining whether the one or more historical device parameters include a historical device parameter matching with the device parameter; and
in response to determining that the one or more historical device parameters include a historical device parameter matching with the device parameter, determining the reference value of the device parameter based on a historical value of the matched historical device parameter.

5. The method of claim 4, wherein the determining the reference values of the one or more device parameters based on a matching relationship between the one or more historical device parameters and the one or more device parameters further includes:
determining one or more first device parameters having matched historical device parameters among the one or more device parameters and one or more second device parameters having no matched historical device parameters among the one or more device parameters; and
determining the reference values of the one or more second device parameters based on the reference values of the one or more first device parameters.

6. The method of claim 5, wherein the determining the reference values of the one or more second device parameters based on the reference values of the one or more first device parameters includes:
obtaining an association model, the association model reflecting a correlation between the one or more first device parameters and the one or more second device parameters, the association model being a machine learning model; and
determining the reference values of the one or more second device parameters by processing the reference values of the one or more first device parameters using the association model.

7. The method of claim 3, further comprising:
selecting a target reference user from at least two reference users based on the user information, wherein each of the at least two reference users has received a historical scan performed by a historical imaging device; and
determining the historical scan of the target reference user as the reference historical scan.

8. The method of claim 3, further comprising:
obtaining, based on the user information, a historical scan of the user performed by a reference imaging device; and
determining the historical scan of the user as the reference historical scan.

9. The method of claim 2, wherein the reference values of the one or more device parameters include a plurality of sets of reference values, and
the determining the target values of the one or more device parameters based on the reference values includes:
determining a recommendation degree of each set of the plurality of sets of reference values; and
determining the target values of the one or more device parameters based on the recommendation degree of each set of the plurality of sets of reference values.

10. The method of claim 9, wherein:
the plurality of sets of reference values include a first set of reference values and a second set of reference values, the first set of reference values is determined based on historical values of one or more historical device parameters used in a reference historical scan, and the second set of reference values is determined based on a parameter prediction model, the parameter prediction model being a machine learning model;
the recommendation degree of the first set of reference values is determined based on a matching relationship between the one or more historical device parameters and the one or more device parameters,
the recommendation degree of the second set of reference values is determined based on a count of usage of the second set of reference values by target reference users in target devices.

11. The method of claim 2, wherein the reference values of the one or more device parameters include a plurality of sets of reference values, and
the determining the target values of the one or more device parameters based on the reference values includes:
sending the plurality of sets of reference values to a user terminal; and
determining one set of the plurality of sets of reference values as the target values based on a selection instruction received from the user terminal.

12. The method of claim 1, wherein the user identifier includes at least one of a numeric identifier or a biometric identifier.

13. The method of claim 12, wherein the numeric identifier includes at least one of a quick response (QR) code or a bar code; or
the biometric identifier includes at least one of a fingerprint, a face, a voice, or an iris.

14. A system for configuring imaging parameters, comprising:
at least one storage device storing a set of instructions; and
at least one processor configured to communicate with the at least one storage device, wherein when executing the set of instructions, the at least one processor is configured to direct the system to perform operations including:
obtaining a user identifier of a user to be examined and one or more device parameters to be set of an imaging device;
determining target values of the one or more device parameters based on the user identifier; and
performing parameter configuration on the imaging device based on the target values of the one or more device parameters.

15. The system of claim 14, wherein the determining target values of the one or more device parameters based on the user identifier comprises:
obtaining user information relating to the user based on the user identifier;
obtaining reference values of the one or more device parameters based on the user information; and
determining the target values of the one or more device parameters based on the reference values.

16. The system of claim 15, wherein obtaining reference values of the one or more device parameters based on the user information comprises:
determining historical values of one or more historical device parameters used in a reference historical scan based on the user information; and
determining the reference values of the one or more device parameters based on a matching relationship between the one or more historical device parameters and the one or more device parameters.

17. The system of claim 15, wherein the reference values of the one or more device parameters includes a plurality of sets of reference values, and
the determining the target values of the one or more device parameters based on the reference values comprises:
determining a recommendation degree of each set of the plurality of sets of reference values; and
determining the target values of the one or more device parameters based on the recommendation degree of each set of the plurality of sets of reference values.

18. The system of claim 17, wherein:
the plurality of sets of reference values include a first set of reference values and a second set of reference values, the first set of reference values is determined based on historical values of one or more historical device parameters used in a reference historical scan, and the second set of reference values is determined based on a parameter prediction model, the parameter prediction model being a machine learning model;
the recommendation degree of the first set of reference values is determined based on a matching relationship between the one or more historical device parameters and the one or more device parameters,
the recommendation degree of the second set of reference values is determined based on a count of usage of the second set of reference values by target reference users in target devices.

19. The system of claim 15, wherein the reference values of the one or more device parameters include a plurality of sets of reference values, and
the determining the target values of the one or more device parameters based on the reference values comprises:
sending the plurality of sets of reference values to a user terminal; and
determining one set of the plurality of sets of reference values as the target values based on a selection instruction received from the user terminal.

20. A non-transitory computer readable medium, comprising a set of instructions, wherein when executed by at least one processor, the set of instructions direct the at least one processor to effectuate a method, the method comprising:
obtaining a user identifier of a user to be examined and one or more device parameters to be set of an imaging device;
determining target values of the one or more device parameters based on the user identifier; and
performing parameter configuration on the imaging device based on the target values of the one or more device parameters.
